(19) United States
(12) Patent Application Publication
(10) Pub. No.: US 2022/0187272 A1
(43) Pub. Date: Jun. 16, 2022

(54) PROCESS AND SYSTEM FOR MEASURING THE OXIDATION STABILITY AND/OR THE THERMAL STABILITY OF A FUEL

(71) Applicant: IFP Energies nouvelles, RUEIL-MALMAISON CEDEX (FR)

(72) Inventors: Maira ALVES FORTUNATO, RUEIL-MALMAISON CEDEX (FR); Arij BEN AMARA, RUEIL-MALMAISON CEDEX (FR); Remy MINGANT, RUEIL-MALMAISON CEDEX (FR); Valerie SAUVANT-MOYNOT, RUEIL-MALMAISON CEDEX (FR); Laurie STARCK, RUEIL-MALMAISON CEDEX (FR); Sofiane BELAID, RUEIL-MALMAISON CEDEX (FR)

(21) Appl. No.: 17/436,282

(22) PCT Filed: Mar. 26, 2020
(86) PCT No.: PCT/EP2020/058557

§ 371 Date: Sep. 3, 2021

(30) Foreign Application Priority Data
Apr. 9, 2019 (FR) FR1903777

(51) Int. Cl. G01N 33/28; G01N 27/02

(52) U.S. Cl. CPC G01N 33/2805; G01N 27/026

(57) ABSTRACT

The present invention relates to a system for characterizing at least one of the oxidation stability and the thermal stability of a fuel sample. The system comprises at least a device (MSIE) for performing an electrochemical impedance spectroscopy measurement in the sample, and a device (MDM) for determining deposit mass forming in the fuel sample. The invention further relates to a method for characterizing at least one of the oxidation stability and the thermal stability of the fuel over time, from measurements performed by the system according to the invention.

### CROSS-REFERENCE TO RELATED APPLICATIONS

Reference is made to PCT/EP2020/058557 filed Mar. 26, 2020, and French Patent Application No. 1903777 filed Apr. 9, 2019, which are incorporated herein by reference in their entirety.

### BACKGROUND OF THE INVENTION

### Field of the Invention

The present invention relates to fuel analysis and characterization, notably for ground transportation or aviation fuel. It more specifically relates to at least one method and system allowing measurement and monitoring of at least one of the oxidation stability and the thermal stability of the fuel.

### Description of the Prior Art

The stability of fuels generates considerable interest in the field of aeronautics and land vehicles.

In the automotive field emission standards imposed on vehicles motivate manufacturers to develop increasingly efficient engines in order to reduce emissions at source from combustion which results in a current combustion engine technology with increasingly stringent operating conditions for diesel injection systems which increases thermal stresses (T>150° C.), a pressure increases (P>2500 bar), combined with a decrease in the diameter of the injector ports.

In the aeronautical field, the temperature and pressure rise cycles imposed on the fuel, combined with increasingly severe conditions, are more and more critical for maintaining fuel stability.

In parallel, the evolution of the environmental, economic and regulatory concerns has promoted the emergence of biofuels and alternative fuels in ground and air transportation. Now, the degradation of these fuels by oxidation can generate the formation of insoluble products and deposits of various origins and compositions in tanks, fuel circuits, combustion systems and injection circuits. These deposits can cause mechanical malfunctions of the pumps and injectors, as well as filter clogging and thus increased pressure drops. It is therefore essential to be able to accurately predict the oxidation state of a fuel to limit such phenomena and when possible prevention.

The degradation of biofuels and alternative fuels in ground transportation concerns both biofuels of fatty acid methyl ester type (FAME) and biofuels of hydrotreated vegetable oil (HVO) type. In the case of aviation jet fuels, oxidation stability concerns all the sectors (HEFA or ATJ-SPK for example).

Thus, there is an increasing demand notably in the automotive or the aviation industry, for a thermal stability and oxidation stability characterization more representative of the new stresses undergone by fuels to avoid problems caused by fuel degradation and logistics refining which in particular are deposition-related problems.

The following documents are mentioned in the description hereafter:
Alves Fortunato, M., Lenglet, F., Ben Amara, A., and Starck, L., “Are Internal Diesel Injector Deposits (IDID) Mainly Linked to Biofuel Chemical Composition or/and Engine Operation Condition?,” SAE Technical Paper 2019-01-0061, 2019, https://doi.org/10.4271/2019-01-0061.
De Souza, E., Scherer, M. D., Ciceres, J. A. S., Caires, A. R. L., M'Peko, J.-C., “A Close Dielectric Spectroscopic Analysis of Diesel/Biodiesel Blends and Potential Dielectric Approaches for Biodiesel Content Assessment”, Fuel, Volume 105, 2013, Pages 705-710, ISSN 0016-2361, https://doi.org/10.1016/j.fuel.2012.09.032.

Known methods of monitoring automotive fuel standards uses measurement of the induction period (one may refer to the ASTM D525 or ASTM D7545 standard methods, or to the EN 15751 standard) or monitoring the formation of liquid insolubles (the ASTM D2274 standard method). However, these techniques are not representative of the degradation of fuels in real systems because some fuels have a very long induction period (therefore are a priori very stable) may indeed degrade very rapidly in an injection system, as described for example (Alves Fortunato et al., 2019). Furthermore, these are laboratory techniques that do not enable in-situ detection of the oxidation tendency of the fuel.

In the case of the aviation industry, the thermal stability of jet fuels is controlled and measured through the JFTOT™ test (Jet Fuel Thermal Oxidation Tester) with an example being described in the ASTM D3241 standard method. The JFTOT™ test circulates jet fuel in a heated tube. This equipment involves the drawback of being bulky and of difficult interpretation. Moreover, the JFTOT™ test is not representative of problems encountered by the industry and it is not discriminating enough between the various kerosenes available on the market.

Published patent application PCT/EP2018/073,335 relates to a device for measuring at least one of the oxidation stability and the thermal stability of any type of fuel, including diesel fuel, by miniaturization of the test system by a microfluidic technique. The physical phenomena undergone by fuels are reproduced by the microchannels of the microfluidic chip, which are representative of at least one of the fuel injection and the fuel circulation for a drive system, an internal-combustion engine or an aircraft reactor. However, this method does not allow the mass of the deposits formed to be measured.

Thus, there is currently no method capable of indicating the various stages of the formation of oxidation products in a liquid phase of insoluble deposits in a reliable manner, representative of the real instability phenomena encountered in fuel systems, whether automotive or aviation fuel systems.

### SUMMARY OF THE INVENTION

The present invention overcomes these drawbacks. In particular, the present invention relates to a method and to a system for monitoring the tendency of a fuel to degrade and to form a deposit, and thus take preventive action.

The present invention relates to a system for determining at least one of the oxidation stability and the thermal stability of a fuel which comprises at least:
a) a device for performing an electrochemical impedance spectroscopy measurement in the fuel; and
b) a device for determining a deposit mass forming in the fuel.

According to one implementation of the invention, said device for determining a mass of a deposit can be a piezoelectric microbalance.

According to one implementation of the invention, the piezoelectric microbalance can be a piezoelectric quartz microbalance.

According to one implementation of the invention, the device for performing an electrochemical impedance spectroscopy measurement can comprise at least one frequency response analyser.

According to one implementation of the invention, the device for performing an electrochemical impedance spectroscopy measurement can comprise at least one interdigitated electrode sensor.

According to one implementation of the invention, the system can further comprise means for automating the measurements carried out by the device for performing at least one of an electrochemical impedance spectroscopy measurement and by the device for determining a mass of a deposit.

According to one implementation of the invention, the system can further transmit at least one of the measurements and means for processing the measurements.

According to one implementation of the invention, the means for processing the measurements make possible, from a measurement carried out by the device for performing an electrochemical impedance spectroscopy measurement, to make a spectral analysis for determining values of the components of an equivalent electrical representation of the sample, with components of the equivalent electrical representation comprising at least a first resistor in series with a capacitor with the capacitor being in parallel with a second resistor.

The invention further relates to a method for determining at least one of the oxidation stability and the thermal stability of a fuel over time wherein, from at least one sample of the fuel, at least steps are carried out for each time interval of plural time interval steps of:
i) performing electrochemical impedance spectroscopy measurements on the sample and measurements of the mass variation of a deposit formed in the sample, by the system described above;
ii) from the electrochemical impedance spectroscopy measurements, determining values of components of an equivalent electrical representation of the sample, the components of the equivalent electrical representation comprising at least a first resistor in series with a capacitor with the capacitor being in parallel with a second resistor; and
iii) from at least part of the values of the components of the equivalent electrical representation of the sample and the measurements of mass variation of the deposit for the time interval, at least one of the oxidation stability and thermal stability of the sample of the fuel for the time interval.

According to one implementation of the invention, step iii) can be carried out by comparing at least part of the values of the components of the equivalent electrical representation of the sample for at least one of time interval and the values of the measurements of the mass variation of the deposit for the time interval with predetermined reference threshold values.

According to one implementation of the invention, step iii) can be applied by use of at least a first reference threshold value for capacitance of the capacitor of about 6.10-¹¹ F and of a second reference threshold value for the capacitance of the capacitor of about 1.10-¹⁰ F, wherein the first reference threshold value for the capacitance is an indicator of a start of oxidation of the fuel sample and the second reference threshold value for the capacitance is an indicator of a start of deposition in the sample.

According to one implementation of the invention, step iii) can be applied by use of at least a first reference threshold value for the second resistor defined by R1-init/2 and of a second reference threshold value for the second resistor defined by R1-init/5, where R1-init is a value of the second resistor determined for the first time interval, the first reference threshold value for the second resistor is an indicator of a start of oxidation of the fuel sample and the second reference threshold value for the second resistor is an indicator of a start of deposition in the sample.

According to one implementation of the invention, step iii) can be applied by use of at least a first reference threshold value for the first resistor defined by R0-init/2 and of a second reference threshold value for the first resistor defined by R0-init/5, where R0-init is a value of the first resistor determined for the first time interval, the first reference threshold value for the first resistor is an indicator of a start of oxidation of the fuel sample and the second reference threshold value for the first resistor is an indicator of a start of deposition of the sample.

According to one implementation of the invention, step iii) can be applied by use of at least one reference threshold value for the mass variation of the deposit of about 15 μg/cm² as an indicator of a deposition start in the sample.

According to one implementation of the invention, prior to step iii), the reference threshold values can be predetermined by use of a reference fuel sample and of at least a reference method for characterizing at least one of the oxidation stability and the thermal stability of a fuel.

### BRIEF DESCRIPTION OF THE DRAWINGS

Other features and advantages of the invention are clear from reading the description hereafter of embodiments given by way of non-limitative example, with reference to the accompanying drawings wherein:

FIGS. 1A and 1B illustrate a distribution of the field lines between a negative electrode and a positive electrode respectively in a form of combs and in a form of plates;

FIGS. 2A and 2B schematically illustrate the equivalent electrical representation between respectively a simple dielectric fluid located between two electrodes and a dielectric fluid deposited on the surface of at least one electrode;

FIG. 3 illustrates an example of a Nyquist diagram showing the imaginary part of the electrochemical impedance measured as a function of the real part thereof,

FIG. 4 illustrates a non-limitative example of a system according to a variant of the invention;

FIGS. 5A, 5B and 5C show the variations over time respectively of the values of resistances R0, R1 and of the capacitance C1 of an electrical equivalent amount determined from measurements performed by the system according to the invention on a fuel sample of BO diesel type;

FIG. 6 shows the mass variation over time resulting from measurements performed by the system according to the invention on the fuel sample of FIGS. 5A to 5C;

FIG. 7 shows evolution of an induction period value over time, resulting from measurements performed with a device according to the prior art;

FIGS. 8A, 8B and 8C show the variations over time respectively of values of resistances R0, R1 and of capacitance C1 of an electrical equivalent amount determined from measurements performed by means of the system according to the invention on a fuel sample of HVO biodiesel type,

FIG. 9 shows the mass variation over time resulting from measurements performed by the system according to the invention on the fuel sample of FIGS. 8A to 8C;

FIGS. 10A, 10B and 10C show variations overtime respectively of values of resistances R0, R1 and of capacitance C1 of an electrical equivalent amount determined from measurements performed by the system according to the invention on a fuel sample of aviation fuel type; and

FIG. 11 shows mass variation over time resulting from measurements performed by the system according to the invention on the fuel sample of FIGS. 10A to 10C.

### DETAILED DESCRIPTION OF THE INVENTION

The present invention relates to a method and to a system for determining at least one of the oxidation stability and the thermal stability of a fuel, from a sample of the fuel to be studied.

According to the invention, the fuel of the sample to be studied can be any type of fuel, including ground and aviation fuels (jet fuel, diesel fuel, gasoline), ground and aviation biofuels included.

The system and the method according to the invention comprise the combination of electrochemical impedance spectroscopy measurements in a fuel sample and of measurements of the mass of a deposit formed in this fuel sample. This combination of different measurements allows reliable determination of the evolution of the changes in the electrochemical state of a fuel sample subjected to at least one of oxidation and to temperature rise, and thus enables early detection (in advance) of the formation of a deposit in this sample. In other words, this combination of different measurements enables detection of starting of deposition in a fuel sample.

The system according to the invention comprises at least:
a device for performing an electrochemical impedance spectroscopy measurement in a fuel to be studied, from a sample of the fuel; and
a device for determining a mass variation of a deposit forming in this fuel, from a sample of this fuel.

Advantageously, the system according to the invention is configured in such a way that the two types of measurement carried out by the device for performing an electrochemical impedance spectroscopy measurement and by use of the device for determining a mass variation of a deposit are simultaneous or, in other words, synchronous. This guarantees coherent interpretation of the two types of measurement performed by the system according to the invention.

In general, electrochemical impedance spectroscopy (EIS) is a technique of analysing the dynamic behavior of an electrochemical system, which applies an electrical perturbation to the system as a function of time and monitors its response over time. More specifically, if an electrochemical system is subjected to an electrical voltage, the current response of the system will reflect the polarization mechanisms involved in the material and the charge transfer phenomena of at least one of the material/electrode interfaces. Generally, electrochemical impedance and permittivity are complex quantities that describe the ability of the material to polarize under the influence of an electric field.

According to one implementation of the invention, the device for performing electrochemical impedance measurements that is used for implementing the method according to the invention comprises at least one of a frequency response analyser connected to at least two electrodes. According to one implementation of the invention, the frequency response analyser is a Solartron 1260 marketed by the Ametek SI company (USA). Advantageously, the Solartron 1260 frequency response analyser can be supplemented by the 1296 dielectric interface marketed by the Ametek SI company (USA), which improves up to 106 times the signal. Such a device allows measurement by electrochemical impedance spectroscopy very high impedances up to 1014Ω. The frequency range available with such an example of a frequency response analyser extends from 10 μHz to 30 MHz.

Advantageously, the frequency range explored by use of the frequency range analyser extends between about 5 Hz and 105 Hz. With fuels which are rather poor electrical conductive fluids (classified among the so-called “dielectric” materials), such a limited frequency range, which is sufficient for monitoring dipolar polarizations, ionic migrations and charge transfer phenomena (at low frequencies), can be advantageously used.

According to one implementation of the invention, the frequency response analyser can be controlled by a computer and of software installed on the computer. According to an implementation of the invention wherein the frequency response analyser is the Solartron 1260, the software can be the SMaRT software developed by the High Tech Detection Systems company (France).

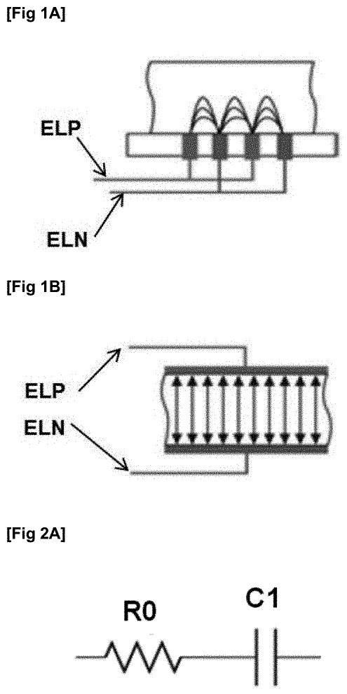

According to one implementation of the invention, the electrodes of the device for performing electrochemical impedance spectroscopy measurements correspond to an interdigitated electrode sensor. This type of sensor is particularly suitable for dielectrometry measurements on poorly conductive materials such as fuels. The interdigitated sensor has a set of positive and negative electrodes arranged in interlocking combs (in other words, the interdigitated sensor is made up of an alternation of positive and negative electrodes). The electrodes are deposited on an insulating substrate and extended by two electrical wires providing connection to a potentiostat. In the case of such an interdigitated electrode sensor, an electric field is created between a positive electrode and a negative electrode. FIG. 1A illustrates such a field line distribution between a negative electrode ELN and a positive electrode ELP in form of combs. When a material is placed on the sensor, subjected to an electrical potential, an electric field flows therethrough and results in a polarization within the material and at the interface; charge transfers may also occur at the interfaces. The dielectric properties of the material and the electrochemical reactions at the interfaces induce the current response between the two electrodes, which allows to determination of the impedance and the permittivity by use of the frequency response analyser.

According to one implementation of the invention, the interdigitated sensor is of the IDEX Micron MS-25/60HT type marketed by the Netzsch company, wherein the electrodes are made of nickel, arranged 25 μm apart and deposited on a quartz substrate.

Alternatively, a parallel plate capacitor, known as planar capacitor, can be used instead of an interdigitated electrode sensor. The operating principle of a parallel plate capacitor is comparable to that of an interdigitated electrode sensor except that, in this case, the distribution of the field lines is parallel. FIG. 1B illustrates such a field line distribution, between a negative electrode ELN and a positive electrode ELP formed in plates.

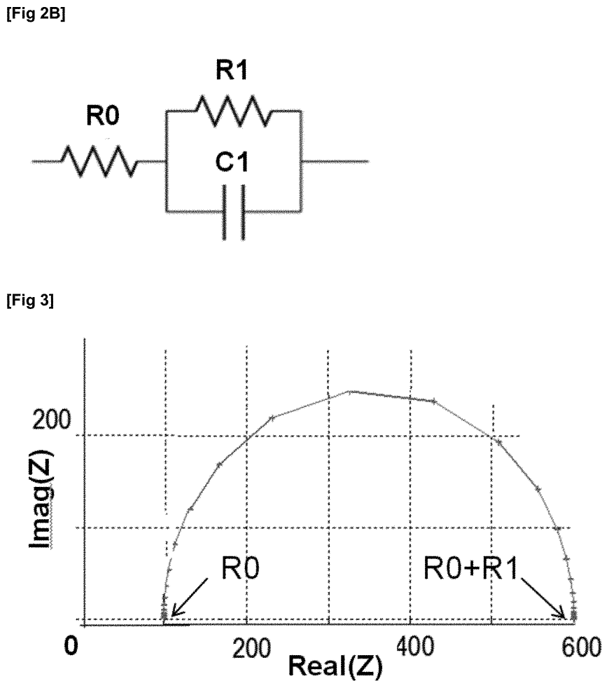

According to the invention, a spectral analysis applied to the electrochemical impedance spectroscopy measurements determines an equivalent electrical representation of the fuel sample with this equivalent electrical representation comprising at least a first resistor in series with a capacitor and the capacitor is in parallel with a second resistor. In general, the EIS spectra of all liquids can be modelled by an equivalent electrical circuit comprising resistors and capacitors. Complements may be found on a spectral analysis example in order to determine an electrical equivalent of a liquid in the document (De Souza et al., 2013). FIGS. 2A and 2B schematically show the equivalent electrical representation between respectively a simple dielectric fluid located between two electrodes and the same dielectric fluid which has a deposit on the surface of at least one electrode. According to this last equivalent electrical representation, R0 is the resistance related to the charge transfer and C1 is a capacitance representing an indicator proportional to the dielectric constant of the fluid studied. It can be noted that, according to an implementation of the invention, the equivalent electrical representation of a fuel sample that has not yet undergone any electrochemical reaction can be modelled by the equivalent electrical representation comprising the components according to FIG. 2B with resistance R1 having in this case an infinite value.

According to one implementation of the invention, the spectral analysis can determine a diagram (called Nyquist diagram) as shown in FIG. 3, which represents the imaginary part of the electrochemical impedance Imag(Z) measured as a function of its real part Real(Z). Such a diagram makes it possible to determine the value of resistances R0 and R1 from the intersections of this diagram with the axis of the zero imaginary values, and the value of capacitance C1 from an equation of the type:

$$C 1 = \frac{1}{{R}_{1} \cdot 2 π {f}_{m ax}}$$

where fₘₐₓ is the bias frequency at the top of the semi-circle of the Nyquist diagram.

According to the invention, a mass variation of a deposit formed in the sample of the fuel to be studied is determined by use of at least one piezoelectric microbalance. A piezoelectric microbalance allows measurement of very low masses by measuring the change in resonance frequency of a piezoelectric crystal. More precisely, in a piezoelectric microbalance, a piezoelectric stimulation is applied via electrodes to a piezoelectric crystal, which then undergoes oscillation. The resonance frequency of the crystal is correlated to the mass thereof with any mass variation of the crystal (generated by a deposit on the crystal for example) causing a variation in the resonance frequency of this crystal. The mass variation can be determined from the frequency variation measurement, for example by use of a formula as follows, based on the Sauerbrey relation:

$$Δ {f}_{z} = - \frac{2 {f}_{0}^{2}}{n A \sqrt{{μ}_{q} {ρ}_{q}}} Δ m$$

where n is the number of the oscillation harmonic of the crystal, f₀ is the resonance frequency of the crystal (in Hz), μ_{q} is the shear modulus of the crystal and ρ_{q} is the density of the crystal, A is the piezoelectric surface of the crystal (area between electrodes, in cm²), and Δfs and Δm are the variations in frequency (in Hz) and in mass (in g) respectively.

Preferably, the piezoelectric microbalance used for implementing the method according to the invention is a quartz crystal microbalance (or QCM), also referred to as quartz microbalance. Indeed, quartz is characterized by an excellent quality factor.

According to one implementation of the invention, a quartz microbalance such as the eQCM 10M model marketed by the GAMRY Instruments company (USA) is used. According to an implementation of the invention, the piezoelectric microbalance is coupled with a potentiostat capable of working in an aqueous medium, such as the model marketed under reference 600 by the GAMRY Instruments company (USA). A potentiostat allows a potential or a current (which may be variable or not) to be applied to the electrode terminals, here the electrodes of the microbalance.

Advantageously, the electrodes of the device for determining the mass variation of a deposit formed in the sample of the fuel to be studied are also used for the device for performing electrochemical impedance spectroscopy measurements. In other words, the electrodes of the system according to the invention are common to the device for determining the mass variation of a deposit formed in the sample of the fuel to be studied and to the device for performing electrochemical impedance spectroscopy measurements.

According to one implementation of the invention, the system according to the invention can further comprise automation use of the measurements to be carried out by the device for performing electrochemical impedance spectroscopy measurements and the device for determining a mass of a deposit formed in this fuel sample. In other words, the electrochemical impedance spectroscopy and deposit mass measurement devices can be controlled by an automaton. This automaton allows preprograming the measurements to be performed and can notably allow them to be synchronized. The automaton, can for example, allow defining a sequencing of the electrochemical impedance spectroscopy measurements to be carried out, simultaneously with the mass measurements of a deposit.

According to one implementation of the invention, the device can further comprise a transmitter for transmitting (by electrical wire, by optical fiber or by wireless transmission for example) the measurements carried out by at least one of the device for performing electrochemical impedance spectroscopy measurements and by at least one of the device for determining a mass of a deposit formed in the fuel sample, and a processor for processing (of a computer using a microprocessor for example) the measurements carried out by the device for performing electrochemical impedance spectroscopy measurements and by the device for determining mass of a deposit formed in this fuel sample.

According to one implementation of the invention, the measurement processor can comprise a computer on which at least one spectral analysis method for determining an equivalent electrical representation of the fuel sample being studied from the electrochemical impedance spectroscopy measurements is implemented. Advantageously, a method for jointly analysing the evolution over time of the values of the components of the equivalent electrical representation of the sample studied and of the mass variation values of a deposit in the sample is also implemented on this computer. The joint analysis can notably aim to determine if predefined thresholds above which a deposit forms in the sample have been reached. In this case, the measurement processor may also comprise a signaling device for signaling when a deposit is forming in the sample, such as an audible or visual alarm, or any type of alarm means.

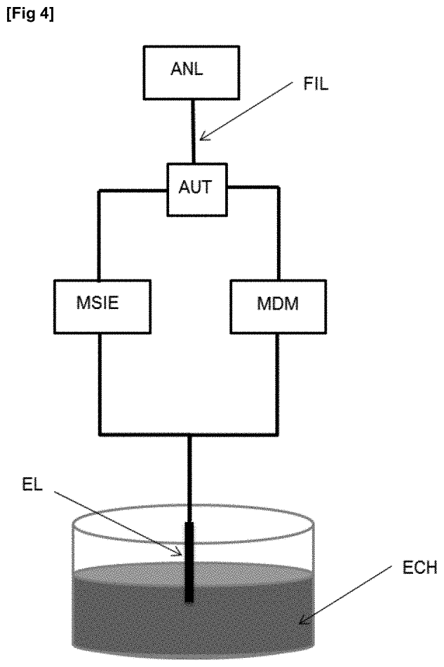

FIG. 4 illustrates a non-limitative example embodiment of the system according to the invention for characterizing at least one of the oxidation stability and the thermal stability of a fuel sample ECH, comprising at least a device MSIE for performing an electrochemical impedance spectroscopy measurement in the sample, a device MDM for determining a mass of a deposit forming in the fuel sample including the MSIE and MDM sharing the same electrode EL. Besides, according to this implementation of the invention, the two devices MSIE and MDM are controlled by an AUT for automating the measurements performed by these two devices, automation device AUT being further connected for example via a wire connection FIL to measurement processor.

According to another aspect, the invention relates to a method for characterizing the oxidation stability and the thermal stability of a fuel. The method according to the invention is implemented from at least one sample of the fuel to be studied and it comprises at least three steps that are each applied for a time interval of time intervals. These time intervals can for example evenly or unevenly split a period during which at least one of the oxidation stability and the thermal stability of the fuel sample is to be monitored.

The method according to the invention comprises at least the following steps for a given time interval:
i) performing an electrochemical impedance spectroscopy measurement on the fuel sample and a measurement of the mass variation of a deposit formed in this sample, by a system for determining at least one of the oxidation stability and the thermal stability of a fuel sample according to any one of the variants described above;
ii) from the electrochemical impedance spectroscopy measurements, determining values of the components of an equivalent electrical representation of the fuel sample, as described above, comprising at least a first resistor in series with a capacitor which the capacitor being in parallel with a second resistor; and
iii) from at least part of the values of the components of the equivalent electrical representation of the fuel sample and the measurements of the mass variation of the deposit for the time interval considered determining at least one of the oxidation stability and the thermal stability of the fuel sample for this time interval.

According to the invention, steps i) to iii) are repeated for each time interval splitting the time period during which at least one of the oxidation stability and the thermal stability of the fuel sample being studied is to be monitored.

In general, as evidenced in the application examples below, the electrochemical impedance measurement provides a qualitative approach to changes that occur within a fluid, while the deposit mass variation measurement permits quantification of a deposit and thus monitoring the fouling kinetics of real equipment. In particular, the combination of the analysis of the electrochemical impedance measurements and of the mass variation measurements allows early detection of a start of aging of a fuel sample, which would not be reliably possible with only one of the two measurement types taken individually. In particular, the impedance measurement considered alone could lead to interpretation errors whereas, when combined with the deposit mass variation measurement, the interpretation becomes more reliable, as shown in the application examples hereafter.

According to one implementation of the invention, step iii) can be applied comparing at least one of at least part of the values of the components of the equivalent electrical representation of the sample and the values of the mass variation measurements of the deposit in the sample with predetermined reference threshold values.

According to one implementation of the invention, the reference threshold values are predetermined by at least one of a reference fuel sample and of at least one of at least one reference method for characterizing the oxidation stability and the thermal stability of a fuel sample. In other words, the reference threshold values are calibrated to a fuel sample, preferably of the same type as that to be monitored, and one or more methods allowing to determinate at least one of the oxidation stability and the thermal stability are applied for each time interval, in addition to steps i) to iii) described above. Among various reference methods, it is possible to select, for example, the induction period measurement method (the ASTM D525 or ASTM D7545 standard methods, or to at least one of the EN 15751 standard) and the ASTM D2274 standard method for monitoring the formation of liquid insolubles.

According to one implementation of the invention, these predetermined threshold values can then be used for early detection, for example by using any variant of the system according to the invention, of the formation of a deposit in another sample of a fuel identical or similar to that on which the threshold values have been predetermined.

According to one implementation of the invention, if the value of capacitance C1 increases, for example from a value C1-init of the order of 4.10-11 F, and if:
at a given time interval, the value of capacitance C1 reaches a reference threshold value of about 6.10-11 F, it can be concluded that oxidation of the fuel has started or, in other words, that fouling has started in the fuel sample being considered, or
at a given time interval, if the value of capacitance C1 reaches a reference threshold value of about 1.10-¹⁰ F, it can be concluded that fouling in the fuel sample is at an advanced stage.

According to one implementation of the invention, if the value of resistance R1 (respectively R0) decreases as a function of time from a value R1-init (respectively R0-init), and if:
at a given time interval, the value of resistance R1 (respectively R0) is less than a reference threshold value that is half the value of R1-init (respectively R0-init), it can be concluded that aging of the fuel has started, or
at a given time interval, if the value of resistance R1 (respectively R0) is less than a reference threshold value that is a fifth of R1-init (respectively R0-init), then the formation of a deposit in the sample considered is effective, or even at an advanced stage.

According to one implementation of the invention wherein the value of resistance R0 is significantly less than the value of resistance R1 (what is meant by significantly less is that R0 is less than at least 0.01*R1), the evolution over time of the values of resistance R0 is not taken into account to characterize at least one of the oxidation stability and the thermal stability of the fuel studied.

According to one implementation of the invention, if the value of the mass variation DM is greater than 15 μg/cm2 (ratio of the mass to the surface area of the measuring electrode), that fouling is established.

More preferably, a cross-interpretation of the values taken over time by resistances R0, R1 and mass variation DM is performed in order to determine a reliable state of progress of a deposit in the sample being considered, for example from the aforementioned predetermined reference threshold values. The interest of such a cross-interpretation is shown in the application examples hereafter, in particular in Example 2 for which a reliable conclusion has been drawn by means of such a cross-interpretation.

### EXAMPLES

The advantages of the method and of the system according to the invention are presented hereafter in three comparative application examples.

In these three examples, a fuel is subjected to an artificial aging process via temperature rise and oxidation. In Example 1, the fuel is a BO type diesel fuel. In Example 2, the fuel is a HVO 9263 type biodiesel fuel. In Example 3, the fuel is an aviation fuel.

The experimental protocol used for the three fuel types is described hereafter. A sample of a volume of 500 ml of a fuel to be tested is placed in a three-neck flask, itself placed in 130° C. (for Example 3) or 150° C. (for Examples 1 and 2) thermostatically-controlled oil bath. Air is then injected at a constant flow rate (10 L/h) into the liquid via a bubbler, to control the oxidation conditions.

The method according to the invention is implemented by a system as described above, notably comprising a device for performing an electrochemical impedance spectroscopy measurement (referred to as EIS measurement hereafter) and a frequency response analyser and of an interdigitated electrode sensor, and a device for determining a mass of a deposit, in form of a quartz microbalance (for performing a measurement referred to as EQCM hereafter). The electrodes of these two devices are immersed in the fuel sample to be studied. EIS and EQCM measurements are carried out continuously over time and analysed in real time. Moreover, liquid samples are collected over time for performing a measurement of induction period type, referred to as IP measurement hereafter, according to the prior art. The IP measurements are carried out by use of the Rancimat device for the diesel type fuels (Examples 1 and 2) and of the PetroOxy device (ASTM D7545) for the jet type fuels (Example 3), used according to the EN 15751 standard.

### Example 1

For this example, the fuel is a BO type diesel fuel. The operating conditions of this example are presented in Table 1.

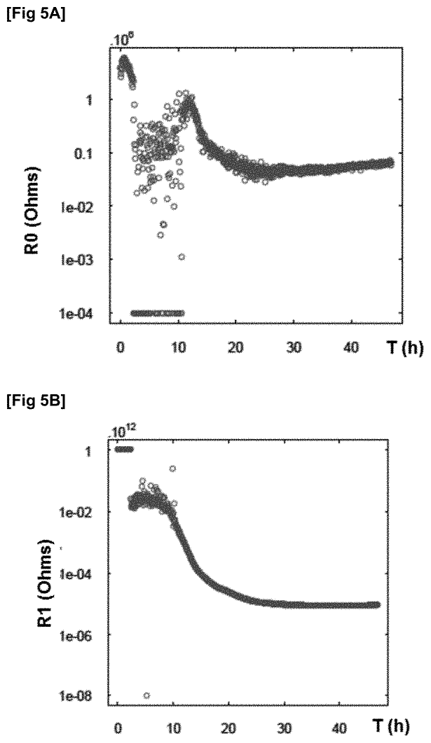

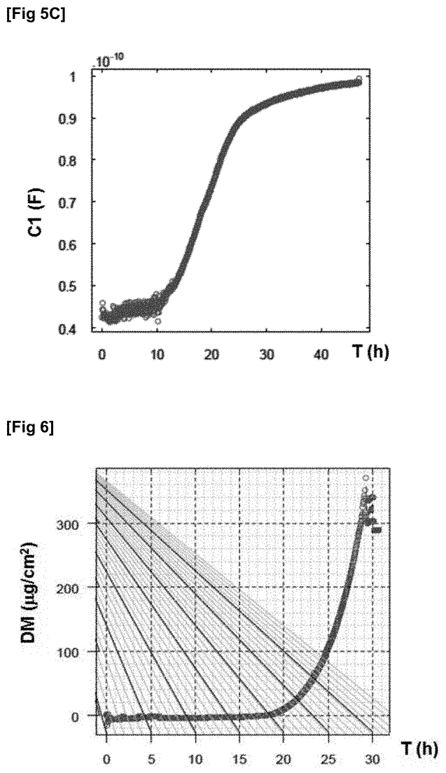

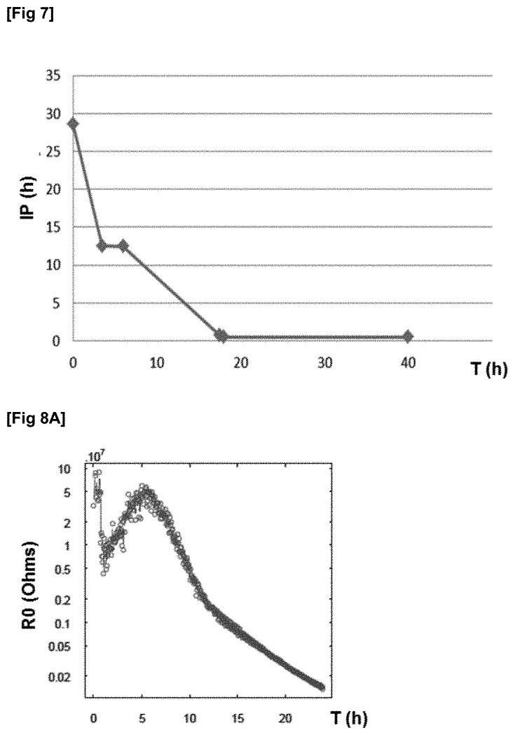

The results of this example are presented in FIGS. 5A, 5B, 5C, FIG. 6 and FIG. 7. FIGS. 5A, 5B and 5C respectively show the variations over time T of resistances R0, R1 and of capacitance C1 of an electrical equivalent determined from the EIS measurements performed on a continuous basis. FIG. 6 shows the mass variation DM over time T resulting from the EQCM measurement performed on a continuous basis. FIG. 7 shows the evolution of the induction period IP as a function of time T, resulting from the continuous IP measurement performed with a device according to the prior art.

By combining the analyses of FIGS. 5A, 5B, 5C and 6, it can be concluded that the fuel studied, subjected to accelerated aging, undergoes four phases:
Phase 1, ranging from 0 to 1 h: This phase corresponds to the temperature rise of the fuel studied. A decrease in resistance R0 can be observed in FIGS. 5A and 5B (as well as a saturation for R1), which is explained by the fact that the resistance of a material generally decreases as a function of temperature. It can also be observed in FIG. 5C that the capacitance is unchanged, which means that no degradation is measured on the sensor surface. It is also noted that no quartz crystal mass gain is observed (unchanged DM values in FIG. 6);
Phase 2, ranging between 1 and 10 h: it is observed in FIG. 6 that the mass gain of the quartz crystal remains negligible, which indicates the absence of deposit. This observation is consistent with the IP values of FIG. 7, which remain high during this phase. The values of R1 (FIG. 5A) and C1 (FIG. 5C) remain globally constant during this phase, but it is noted that resistance R0 tends to decrease significantly (up to the detection limit), which indicates an increase in the number of charge carriers or in the mobility thereof. The fact that capacitance C1 does not increase during this period indicates a stability of the nature of the polarity at the interface. Therefore, the decrease of resistance R0 rather indicates a decrease in the fuel viscosity (related to a chain cleavage);
Phase 3, ranging between 10 and 20 h: it can be observed in FIGS. 5A, 5B and 5C that resistance R0 rises rapidly, then decreases again, while resistance R1 decreases, and C1 increases substantially. In parallel, it is observed that the mass variation measured by the EQCM tends to increase (FIG. 6), even though it remains low. All of these variations, jointly interpreted, mean that the fuel being studied undergoes a chemical change resulting from a chemical reaction (evolutions of R0 and R1), and that a polarity increase at the interface is observed (increase of C1). However, the mass variation of the EQCM being low, it may be concluded that there is no deposit yet at this stage. Besides, this conclusion can be confirmed by the IP measurement, which greatly decreases (see FIG. 7), thus confirming that the product is no longer stable; and
Phase 4, ranging between 20 and 40 h: it can be observed in FIGS. 5A, 5B and 5C that the resistance values are stable, which indicates a constancy in the chemical nature of the medium. On the other hand, a sharp increase in capacitance C1 can be observed, which indicates an evolution of the capacitance at the EIS sensor/electrode interface, and may be a deposit formation indicator. Moreover, it is noted that the EQCM measurement (see FIG. 6) increases considerably, which confirms the interpretation of the increase in capacitance C1 according to which a deposition has occurred in the fuel sample tested. Thus, the combination of the interpretation of the two measurement types, EIS and EQCM, allows to draw a conclusion. It should be noted that the increase of capacitance C1 continues beyond 20 h, indicating the evolution of the capacitance at the sensor/electrode interface, which is consistent with the continuation of deposit formation. The IP values are zero, which confirms that the product is unstable. It is noted that a deposit is visually observed at the end of the test.

TABLE 1
Conditions & protocol

| Volume | 500 | ml |
|---|---|---|
| Temperature | 150° C. | |
| Air flow | 10 | L/h |
| Sampling | V = 15 | ml |

0, 3.5, 6, 17.5, 18 and 40 h
Nitrogen-blanketed flasks
Low-temperature conditioning (<5° C.)

### Example 2

For this example, the fuel is a HVO type (9263) biodiesel fuel. The operating conditions of this example are presented in Table 2.

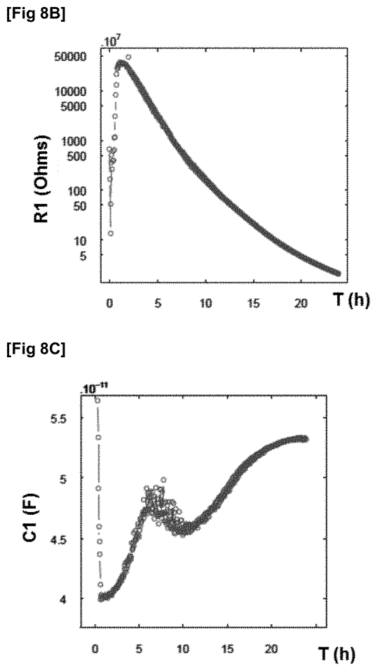

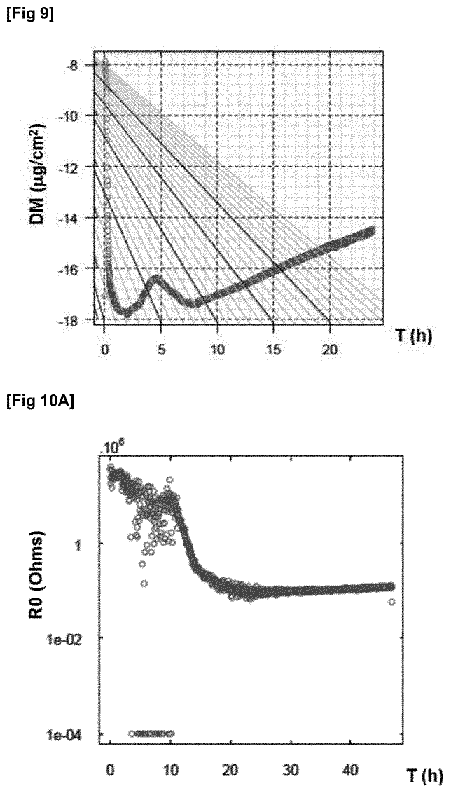

The results of this example are presented in FIGS. 8A, 8B, 8C and 9. FIGS. 8A, 8B and 8C respectively show the variations over time T of resistances R0, R1 and of capacitance C1 of an electrical equivalent determined from the EIS measurements performed on a continuous basis. FIG. 9 shows the mass variation DM over time T resulting from the EQCM measurement performed on a continuous basis.

By combining the analyses of FIGS. 8A, 8B, 8C and 9, it can be concluded that the fuel being studied, subjected to accelerated aging, undergoes four phases:
Phase 1, ranging from 0 to about 1 h: this phase corresponds to the temperature rise of the fuel being studied. A decrease in resistances R0 and R1 can be observed in FIGS. 8A and 8B, which is explained by the fact that the resistance of a material generally decreases as a function of temperature. It can also be observed in FIG. 9 that the EQCM sensor also warms up;
Phase 2, ranging between 1 and 4 h: it is observed in FIGS. 8A, 8B and 8C that resistance R0 increases rapidly, while resistance R1 decreases and capacitance C1 increases substantially. All of these variations mean that the fuel studied undergoes a chemical change resulting from a chemical reaction (evolutions of R0 and R1), and that a polarity increase at the interface is observed (increase of C1). In coherence, it can be seen that a deposit forms according to the EQCM measurement (increase of mass variation DM in FIG. 9);
Phase 3, ranging between 5 and 9 h: it can be observed in FIGS. 8A, 8B and 8C that, while resistances R0 and R1 continue to decrease, C1 decreases sharply, which shows a change in the deposits at the interface. Besides, FIG. 9 shows a decrease in the mass variation determined by the EQCM. By combining the interpretations of both the EQCM and the EIS, it can be concluded that the phenomena observed are to be interpreted as a detachment of the deposit; and
Phase 4, after 10 h: it can be observed in FIGS. 8A, 8B and 8C that the resistance values R0 and R1 continue to decrease, and that C1 resumes its increase. The measurement of the EQCM sensor in FIG. 9 confirms the mass gain and therefore the resumption of the formation of a deposit associated with the ongoing fuel degradation reactions.

TABLE 2
Conditions & protocol

| Volume | 500 | ml |
|---|---|---|
| Temperature | 150° C. | |
| Air flow | 10 | L/h |
| Sampling | V = 15 | ml |

2/day
Nitrogen blanketing
Low-temperature conditioning < 5° C.

### Example 3

For this example, the fuel is an aviation fuel. The operating conditions of this example are presented in Table 3.

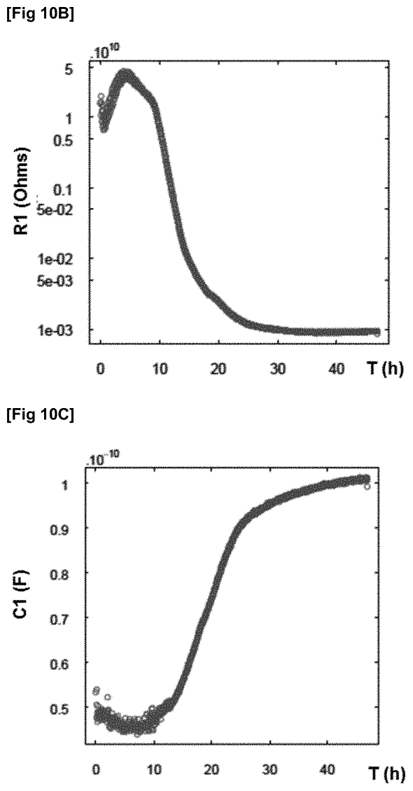

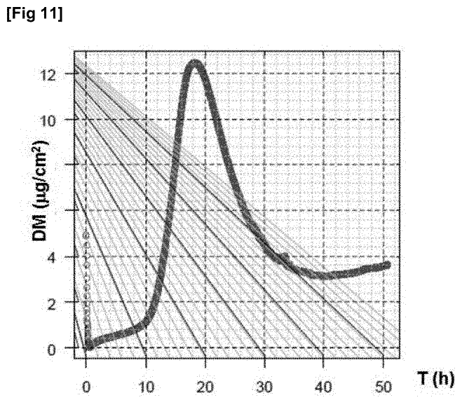

The results of this example are presented in FIGS. 10A, 10B, 10C and 11. FIGS. 10A, 10B and 10C respectively show the variations over time T of resistances R0, R1 and of capacitance C1 of an electrical equivalent determined from the EIS measurements performed on a continuous basis. FIG. 11 shows the mass variation DM over time T resulting from the EQCM measurement performed on a continuous basis.

By combining the analyses of FIGS. 10A, 10B, 10C and 11, it can be concluded that the fuel studied, subjected to accelerated aging, undergoes four phases:
Phase 1, ranging from 0 to 1 h: this phase corresponds to the temperature rise of the fuel studied. A decrease in resistances R0 and R1 can be observed in FIGS. 10A and 10B, which is explained by the fact that the resistance of a material generally decreases as a function of temperature. It can also be observed in FIG. 11 that the EQCM sensor also warms up,
Phase 2, ranging between 1 and 9 h: a plateau can be observed in FIGS. 10A, 10B and 10C for values R0 and C1, while resistance R1 tends to significantly increase (by a factor 5). Besides, it can be seen in FIG. 11 that the mass gain revealed by the EQCM measurement is low, but detectable. The increase of resistance R1 can be interpreted by the formation of a deposit on the comb. The fact that capacitance C1 does not increase during this period may indicate that this deposit does not modify the polarity at the interface, it might therefore be a small deposit. This interpretation is confirmed by the moderate mass gain DM. These conclusions are confirmed by the IP measurement according to the prior art, which decreases sharply, thus indicating a significant stability loss,
Phase 3, ranging between 10 and 18 h: it can be observed in FIGS. 10A, 10B and 10C that resistances R0 and R1 decrease sharply, while capacitance C1 increases. These trends show that the medium becomes more reactive (R1 decreases), and a polarity increase is observed with the increase of C1 (the response of the deposit prevails at the interface). Besides, it is noted that the EQCM measurement greatly increases, from 1 to 12 μg/cm². Cross-interpretation of the EQCM and EIS measurements thus leads to the conclusion that the deposit formation continues,
Phase 4, ranging between 18 and 50 h: it can be observed in FIGS. 10A and 10B that the resistance values are stable. However, FIG. 11 shows that the EQCM measurement decreases and that the deposit tends to decrease until stabilization around 40-50 h. Cross-interpretation of the EQCM and EIS measurements thus leads to the conclusion that the fuel seems to have completed its transformation.

TABLE 3
Conditions & protocol

| Volume | 500 | ml |
|---|---|---|
| Temperature | 130° C. | |
| Air flow | 10 | L/h |
| Sampling | V = 15 | ml |

2/day
Nitrogen blanketing
Low-temperature conditioning < 5° C.

Thus, it clearly appears that the method and the device according to the invention have significant advantages over the IP measurement of the prior art:
the electrochemical impedance measurement provides a qualitative approach to the various changes that occur within the fluid, leading to conclusions similar to that of an IP measurement according to the prior art. The IP measurement can therefore be advantageously replaced by a EIS measurement, more accurate, faster, less expensive and more descriptive; and
the EQCM measurement allows direct quantification of the deposit obtained, and therefore to monitor the fouling kinetics and the fouling rate that would have been observed with a real equipment.

The two types of measurement, EIS and EQCM, are thus complementary; they notably provide qualitative and quantitative information relative to the various mechanisms involved in the aging phase of a fuel.

1.-15. (canceled)

16. A system for determining at least one of oxidation stability and thermal stability of a fuel, comprising:
a) a device for performing an electrochemical impedance spectroscopy measurement in the fuel; and
b) a device for determining a deposit mass forming in the fuel; and
c) a processor for processing the measurement, the processor comprising a computer on which is implemented at least one joint analysis method for determining evolution over time of values of components of an equivalent electrical representation of the fuel determined from the electrochemical impedance spectroscopy measurements and of values of a variation of the deposit mass in the fuel with the at least one joint analysis method enabling detection of a deposit mass of the fuel.

17. A system as claimed in claim 16, wherein the device for determining a mass of the deposit is a piezoelectric microbalance.

18. A system as claimed in claim 17, wherein the piezoelectric microbalance is a piezoelectric quartz microbalance.

19. A system as claimed in claim 16, wherein the device for performing an electrochemical impedance spectroscopy measurement comprises at least one frequency response analyser.

20. A system as claimed in claim 16, wherein the device for performing an electrochemical impedance spectroscopy measurement comprises at least one interdigitated electrode sensor.

21. A system as claimed in claim 16, comprising:
means for automating the measurements carried out by the device for performing at least one of an electrochemical impedance spectroscopy measurement and the device for determining the deposit mass.

22. A system as claimed in claim 16, comprising:
means for transmitting the measurements.

23. A system as claimed in claim 16, wherein the means for processing the measurements performs a spectral analysis to determine values of the components of the equivalent electrical representation of the sample, the components of the equivalent electrical representation comprising at least a first resistor in series with a capacitor in parallel with a second resistor.

24. A method for determining one of oxidation stability and the thermal stability of a fuel over time from at least one sample of the fuel which are carried out for each of multiple time intervals comprising:
i) performing electrochemical impedance spectroscopy measurements on the at least one sample and measurements of the mass variation of a deposit formed in the sample, by use of the system of claim 16;
ii) from the electrochemical impedance spectroscopy measurements, determining values of components of an equivalent electrical representation of the sample, comprising at least a resistor in series with a capacitor with the capacitor being in parallel with another resistor; and
iii) from at least part of the values of the components of the equivalent electrical representation of the sample and the measurements of variation of deposit mass for each of the time intervals with at least one of the oxidation stability and thermal stability of the sample of the fuel being performed for each time interval.

25. A method for determining at least one oxidation stability and thermal stability of a fuel as claimed in claim 24, wherein step iii) is carried out by comparing at least part of the values of the components of the equivalent electrical representation of each sample for the time interval and the values of the measurements of variation of the mass of the deposit for each time interval with predetermined reference threshold values.

26. A method as claimed in claim 25, wherein step iii) is applied by at least a first reference threshold value for the capacitance of the capacitor of about 6.10-¹¹ F and of a second reference threshold value for the capacitance of the capacitor of about 1.10-¹⁰ F, the first reference threshold value for the capacitance being an indicator of a start of oxidation of the fuel sample and the second reference threshold value of the capacitance being an indicator of a start of deposition in the sample.

27. A method as claimed in claim 25, wherein step iii) is applied by at least a first reference threshold value for the second resistor defined by R1-init/2 and of a second reference threshold value for the second resistor defined by R1-init/5, where R1-init is a value of the second resistor determined for the first time interval, the first reference threshold value for the second resistor being an indicator of starting of oxidation of the fuel sample and the second reference threshold value for the second resistor being an indicator of starting of deposition in the sample.

28. A method as claimed in claim 26, wherein step iii) is performed with use of at least a first reference threshold value for a first resistor defined by R0-init/2 and of a second reference threshold value for the first resistor defined by R0-init/5, where R0-init is a value of the first resistor determined for the first time interval with the first reference threshold value for the first resistor being an indicator of the starting of oxidation of the fuel sample and the second reference threshold value for the first resistor being an indicator of starting of deposition in the sample.

29. A method as claimed in claim 25, wherein step iii) is applied with at least one reference threshold value of mass variation of the deposit of about 15 μg/cm² as an indicator of starting of deposition in the sample.

30. A method as claimed in claim 25, wherein, prior to step iii), the reference threshold values are predetermined by use of a reference fuel sample and of at least a reference method for determining at least one of the oxidation stability and thermal stability of a fuel.
